(19) United States
(12) Patent Application Publication
(10) Pub. No.: US 2020/0069843 A1
(43) Pub. Date: Mar. 5, 2020

(54) METHODS FOR PREPARING A DECELLULARIZED MUSCLE SCAFFOLD

(71) Applicant: Colorado State University Research Foundation, Fort Collins, CO (US)

(72) Inventors: Ruth J. Rose, Fort Collins, CO (US); Nicole P. Ehrhart, Fort Collins, CO (US); Matthew E. Randolph, Bronx, NY (US); Laura Chubb, Fort Collins, CO (US)

(73) Assignee: Colorado State University Research Foundation, Fort Collins, CO (US)

(21) Appl. No.: 16/614,602

(22) PCT Filed: May 18, 2018
(86) PCT No.: PCT/US2018/033356

§ 371 Date: Nov. 18, 2019

Related U.S. Application Data
(60) Provisional application No. 62/508,733, filed on May 19, 2017.

(51) Int. Cl. A61L 27/36; A61L 27/38; A61K 35/34; A61K 35/28; C12N 5/00

(52) U.S. Cl. CPC A61L 27/3687; A61L 27/3691; A61L 27/3826; A61K 35/34; C12N 2513/00; A61L 27/3834; C12N 5/0081; A61L 2430/30; A61L 2430/40; A61K 35/28

(57) ABSTRACT

The present disclosure provides a method for preparing a decellularized muscle scaffold, decellularized muscle scaffolds produced therefrom, and uses thereof to treat a subject with damaged or lost muscle.

### CROSS-REFERENCE TO RELATED APPLICATIONS

This application claims priority to U.S. provisional application No. 62/508,733, filed May 19, 2017, which is hereby incorporated by reference in its entirety.

### FIELD OF THE INVENTION

The present disclosure provides a method for preparing a decellularized muscle scaffold, decellularized muscle scaffolds produced therefrom, and uses thereof to treat a subject with damaged or lost muscle.

### BACKGROUND OF THE INVENTION

Techniques to repair structural and/or functional damage to muscle are currently limited and could benefit tremendously from advances in tissue engineering. Recent advances in tissue engineering hold promise for repair of such damage. Three primary tissue engineering technologies exist for the fabrication of such engineered muscle tissue: assembled scaffolds of synthetic polymers, naturally occurring extracellular matrix (ECM) proteins of collagen or fibrin, or micropatterned surfaces; decellularized tissue ECMs; and scaffold-free (or self-assembled) tissue engineering. Despite the many approaches to skeletal muscle tissue engineering, there remains a need in the art for a tissue engineering strategy that integrates readily with endogenous tissue and provides sufficient myogenic regeneration to repair and/or replace damaged or lost muscle tissue.

### SUMMARY OF THE INVENTION

In an aspect, the present disclosure encompasses a method for preparing a decellularized muscle scaffold comprising (a) providing a muscle sample from a donor and treating the muscle sample with an aqueous solution comprising about 0.5% (w/v) to about 2% (w/v) of an anionic detergent for greater than 72 hours at room temperature, with agitation, to produce a treated muscle sample, wherein the aqueous solution is replaced about every 20 to 28 hours; and (b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period.

In another aspect, the present disclosure encompasses a method for preparing a decellularized muscle scaffold comprising (a) providing a muscle sample from a donor and treating the muscle sample with an aqueous solution comprising about 0.5% (w/v) to about 2% (w/v) sodium dodecyl sulfate for greater than 72 hours at room temperature, with agitation, to produce a treated muscle sample, wherein the aqueous solution is replaced about every 20 to 28 hours; and (b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period.

In another aspect, the present disclosure provides a decellularized muscle scaffold produced by a method, the method comprising (a) providing a muscle sample from a donor and treating the muscle sample with an aqueous solution comprising about 0.5% (w/v) to about 2% (w/v) of an anionic detergent for greater than 72 hours at room temperature, with agitation, to produce a treated muscle sample, wherein the aqueous solution is replaced about every 20 to 28 hours; and (b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period.

In another aspect, the present disclosure provides a decellularized muscle scaffold produced by a method, the method comprising (a) providing a muscle sample from a donor and treating the muscle sample with an aqueous solution comprising about 0.5% (w/v) to about 2% (w/v) sodium dodecyl sulfate for greater than 72 hours at room temperature, with agitation, to produce a treated muscle sample, wherein the aqueous solution is replaced about every 20 to 28 hours; and (b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period.

In another aspect, the present disclosure provides a decellularized muscle scaffold produced by a method, the method comprising (a) providing a muscle sample from a donor and treating the muscle sample with an aqueous solution comprising about 0.5% (w/v) to about 2% (w/v) sodium dodecyl sulfate for greater than 72 hours at room temperature, with agitation, to produce a treated muscle sample, wherein the aqueous solution is replaced about every 20 to 28 hours; (b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period; and (c) seeding the decellularized muscle scaffold with a plurality of muscle stem cells.

In another aspect, the present disclosure provides a decellularized muscle scaffold produced by a method, the method comprising (a) providing a muscle sample from a donor and treating the muscle sample with an aqueous solution comprising about 0.5% (w/v) to about 2% (w/v) sodium dodecyl sulfate for greater than 72 hours at room temperature, with agitation, to produce a treated muscle sample, wherein the aqueous solution is replaced about every 20 to 28 hours; (b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period; and (c) treating the decellularized muscle scaffold to attract endogenous muscle stem cells when transplanted in a subject.

In another aspect, the present disclosure provides a method for treating a subject with damaged or lost muscle, the method comprising transplanting a decellularized muscle scaffold of the present disclosure into an area of damaged or lost muscle in a subject.

In another aspect, the present disclosure provides a method for treating a subject with damaged or lost muscle, the method comprising transplanting a decellularized muscle scaffold of the present disclosure into an area of damaged or lost muscle in a subject, wherein the decellularized muscle scaffold is seeded with a plurality of muscle stem cells.

In another aspect, the present disclosure provides a method for treating a subject with damaged or lost muscle, the method comprising transplanting a decellularized muscle scaffold of the present disclosure into an area of damaged or lost muscle in a subject, wherein the decellularized muscle scaffold is treated to attract the subject's endogenous muscle stem cells.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1 is an IVIS image of a transgenic Wistar rat universally expressing luciferase.

FIGS. 2A-2F are images of the surgical technique for creating volumetric muscle defect in a Wistar rat.

FIG. 3 is an image of rat in a tetanic testing system.

FIGS. 4A-4D are images of a muscle sample before and after decellularization. FIG. 4A is an IVIS image of a tibialis anterior (TA) muscle sample before decellularization. Luciferase expression is evident. FIG. 4B is an IVIS image of a TA muscle sample after decellularization. Luciferase expression is absent. FIG. 4C is a 40× micrograph of an H&E stained TA muscle sample after decellularization. There is no longer any nuclei present indicating decellularization. FIG. 4D is a 20× micrograph of H&E staining of a decellularized TA muscle scaffold. There is no longer any nuclei present indicating decellularization.

FIG. 5 is a graph of the DNA content of various TA muscle samples. Abbreviation: “decell”=decellularization.

FIGS. 6A-6B are SEM images of a decellularized TA muscle scaffold showing preservation of the microstructure.

FIG. 7 is an image of luciferase expressing SMdMSCs in culture. The cells are imaged with BLI.

FIGS. 8A-8F are trilineage differentiation images of muscle derived stem cells. (not on scaffold). FIGS. 8A-8C and FIGS. 8D-8F are osteogenic, adipogenic, and chondrogenic images (as labeled) of differentiated muscle derived stem cells from two separate cell isolations.

FIGS. 9A-9G show data from re-cellularization experiments. FIG. 9A is an image of Luc+SMdMSCs on a decellularized muscle scaffold at 96 hours. Seeded scaffolds (red circles) are visualized on the far left wells of the six-well plate. In the far right wells, unseeded scaffolds are present, which do not express the luciferase enzyme (green circles). FIG. 9B is a graph of luciferase expression. Cells on the seeded scaffold show consistently greater bioluminescence (blue line) than the non-seeded scaffold (red line). “Pre cells” refers to the cells just before cell seeding. FIG. 9C is a micrograph of H&E staining of a decellularized TA muscle scaffold 72 hours after being seeded with Luc+SMdMSCs (40× magnification). The cells are distributed superficially (arrows) on the scaffold (asterisks). FIG. 9D-9G are micrographs of immunohistochemical staining of cells on a decellularized TA muscle scaffold 72 hours after seeding. Immunohistochemical staining for luciferase was performed using a DAB stain. Luciferase positive cells (arrows) along the superficial layers of the scaffold (asterisks) stain brown. FIG. 9D is an image without addition of antibody to luciferase as a negative control of staining for luciferase expressing muscle derived stem cells. FIG. 9E is an image of positive staining for luciferase expressing muscle derived stem cells. FIG. 9F is an image of positive staining for luciferase expressing muscle derived stem cells. FIG. 9G is an image of positive staining for luciferase expressing muscle derived stem cells.

FIGS. 10A-10C are representative images of an animal that received Luc+SMdMSCs seeded scaffolds from day one (FIG. 10A), day 7 (FIG. 10B), and day 14 (FIG. 10C) post-surgery.

FIGS. 11A-11I are representative images of animals post-surgery. FIG. 11A and FIG. 11B are representative images of inflammation scores of 1 and 2, respectively. FIG. 11A demonstrates a minimal inflammatory response within the defect characterized by lymphocytes and plasma cells with fewer pigment-laden macrophages (10×). FIG. 11B demonstrates a mild to moderate inflammatory response within the defect predominantly characterized by lymphocytes and plasma cells with fewer pigment-laden macrophages (10×). Overall, little inflammation was seen within the samples and the scoring was developed relative to this study. FIG. 11C and FIG. 11D are representative images of integration scores of 1 and 3, respectively. FIG. 11C demonstrates mildly increased amounts of loosely organized fibrous connective tissue and thin bundles of myocytes, which are short in length, haphazardly arranged and minimally integrated into or filling the muscle defect, as evidenced by increased amounts of clear space within the defect (5×). The image on the right demonstrates markedly increased amounts of dense fibrous connective tissue bands and few bundles of myocytes, but which are arranged in orderly, longitudinal band which completely fill and span the entire length of the defect and have a seamless transition to the underlying normal skeletal muscle (5×). FIGS. 11E-11G are representative images of myogenic response scores of 1, 2 and 3, respectively. FIG. 11E demonstrates mild proliferation of satellite cells in linear arrangements, which surround normal skeletal myocytes and mildly expand the perimysium and endomysium (5×). FIG. 11F demonstrates moderate proliferation of satellite cells in linear to coalescing nodular arrangements, which surround and separate normal skeletal myocytes and moderately expand the perimysium and endomysium (5×). FIG. 11G demonstrates marked regeneration of skeletal myocytes characterized by myocytes with increased sarcoplasmic basophilia, and nuclear internalization and rowing, as well as marked proliferation of satellite cells which surround regenerating myocytes and markedly expand the perimysium and endomysium (5×). FIGS. 11H-11I are representative images of fibrosis scores of 1 and 3, respectively. FIG. 11H demonstrates mildly increased amounts of loosely organized fibrous connective tissue (blue) which attempts to fill the muscle defect and surround skeletal myocytes (5×). FIG. 11I demonstrates markedly increased amounts of dense fibrous connective tissue (blue) organized into dense linear bands which span and fill the muscle defect (5×).

FIGS. 12A-120 are graphs of the means scores of integration (FIG. 12A), inflammation (FIG. 12B), myocyte regeneration (FIG. 12C), and fibrosis (FIG. 12D). The DMS/Luc+SMdMSC group had significantly greater scores of integration, inflammation, myocyte regeneration and fibrosis than the empty group (*). The DMS/Luc+SMdMSC group also had a greater myocyte response than the DMS alone group (*).

FIG. 13 is a graph showing the mean percent (%) difference in peak tetanic force (P₀) between the VML TA (operated) and contralateral TA (un-operated) muscle. Empty defect rates (n=8) recovered, on average, 66% of normal P₀; the DMS rats (n=9) recovered 73% of normal P₀; and DMS/Luc+SMdMSC rats (n=8) recovered 90% of normal P₀.

### DETAILED DESCRIPTION

The present disclosure provides a method for preparing a decellularized muscle scaffold, decellularized muscle scaffolds produced therefrom, and uses of the decellularized muscle scaffolds. A “decellularized muscle scaffold,” as used herein, refers to muscle tissue obtained from a donor (“donor tissue”) that has been physically, chemically, and/or enzymatically treated to decrease the cellular component and DNA content of the donor tissue. The term “decellularized muscle scaffold” includes muscle tissue that is completely decellularized and also muscle tissue that is incompletely decellularized. Complete decellularization results in a scaffold consisting of extracellular matrix (ECM). Incomplete decellularization results in a scaffold comprising extracellular matrix (ECM), and an amount of the cellular component and/or an amount of the DNA content from the donor tissue. An ECM comprises secreted products of the resident cells of the donor tissue. It includes both functional and structural molecules (proteins, carbohydrates, etc.) arranged in a three-dimensional ultrastructure. When a decellularized muscle scaffold of the present disclosure is re-cellularized (for example by transplantation with stem cells), the ECM supports the phenotype and the function of the transplanted cells.

### I. Methods for Preparing a Decellularized Muscle Scaffold

In an aspect, the present disclosure provides a method for preparing a decellularized muscle scaffold. Decellularization can be confirmed microscopically and/or by quantifying residual DNA content of the donor tissue using method well-known in the art, and further detailed in the examples. An advantage of the method of the present disclosure is that the decellularized muscle scaffold produced has a preserved microstructure, for example as determined microscopically (e.g., scanning electron microscopy (SEM), immunohistochemistry+microscopy, etc.). Another advantage of the method of the present disclosure is that the decellularized muscle scaffold supports proliferation and differentiation of progenitor cells. A further advantage of the method of the present disclosure is that the decellularized muscle scaffold produced has a low DNA content—for example, less than 50 ng of DNA per mg of decellularized muscle scaffold. In some embodiments, a decellularized muscle scaffold of the present disclosure has a DNA content of not more than about 45 ng of DNA per mg of decellularized muscle scaffold. In some embodiments, a decellularized muscle scaffold of the present disclosure has a DNA content of not more than about 40 ng of DNA per mg of decellularized muscle scaffold. In some embodiments, a decellularized muscle scaffold of the present disclosure has a DNA content of not more than about 36 ng of DNA per mg of decellularized muscle scaffold. In other embodiments, a decellularized muscle scaffold of the present disclosure has a DNA content of not more than about 34 ng of DNA per mg of decellularized muscle scaffold. In other embodiments, a decellularized muscle scaffold of the present disclosure has a DNA content of not more than about 30 ng of DNA per mg of decellularized muscle scaffold. In other embodiments, a decellularized muscle scaffold of the present disclosure has a DNA content of not more than about 28 ng of DNA per mg of decellularized muscle scaffold. In other embodiments, a decellularized muscle scaffold of the present disclosure has a DNA content of not more than about 26 ng of DNA per mg of decellularized muscle scaffold. In other embodiments, a decellularized muscle scaffold of the present disclosure has a DNA content of not more than about 24 ng of DNA per mg of decellularized muscle scaffold. In certain embodiments, methods of the present disclosure result in decellularized muscle scaffolds that have a preserved microstructure, as determined by SEM, and a DNA content less than 50 ng of DNA per mg of decellularized muscle scaffold and preferably even lower.

In certain embodiments, decellularization can be further confirmed by one or more additional methods. For example, loss of one or more cellular markers of the donor tissue may be followed. In one embodiment, the cellular marker is endogenously expressed by the tissue. In one embodiment, the cellular marker is the product of a transgene expressed by the tissue. Suitable transgenes are known in the art. In one example, the transgene may encode luciferase or any other protein that allows for the use of bioluminescence, chemiluminescence or fluorescence.

Generally, a method of the present disclosure comprises providing muscle tissue from a donor, treating the muscle tissue with an aqueous solution comprising a detergent under suitable conditions to produce a treated muscle sample; and washing the treated muscle tissue with a wash solution under suitable conditions to produce a decellularized muscle scaffold. In at least one example, a method of the present disclosure further comprises adding a sufficient amount of a storage buffer to the decellularized muscle scaffold and storing at about 4° C. In another example, a method of the present disclosure further comprises freezing or lyophilizing (freeze drying) the decellularized muscle scaffold.

Muscle tissue from any suitable donor may be used. Non-limiting examples of suitable donors include mammals, birds, fish, reptiles, and amphibians. Non-limiting examples of suitable mammal donors include a human or a non-human mammal, such as a livestock animal, a companion animal, a lab animal, a zoological animal, etc. In one embodiment, a donor may be a rodent, e.g., a mouse, a rat, a guinea pig, etc. In another embodiment, a donor may be a livestock animal. Non-limiting examples of suitable livestock animals include pigs, cows, horses, goats, sheep, llamas and alpacas. In yet another embodiment, a donor may be a companion animal. Non-limiting examples of companion animals include pets such as dogs, cats, rabbits, etc. In yet another embodiment, a donor may be a zoological animal. As used herein, a “zoological animal” refers to an animal that may be found in a zoo. Such animals include non-human primates, large cats, wolves, bears, etc. In certain embodiments, the animal is a laboratory animal. Non-limiting examples of a laboratory animal include rodents, rabbits, canines, felines, non-human primates, etc. In certain embodiments, a muscle sample is obtained from a mouse, a rat, a human, a cat, a dog, goat, or a sheep. In an exemplary embodiment, the muscle sample is a skeletal muscle sample, optionally a sample from a tibialis anterior muscle or a quadriceps muscle.

The muscle tissue can be a sample of smooth muscle, a sample of cardiac muscle, or a sample of skeletal muscle. Samples of muscle tissue can be obtained by dissection, biopsy, or any other method known in the art. In one non-limiting example, a sample of smooth muscle is obtained from a bladder or an intestine. In another non-limiting example, a sample of skeletal muscle is obtained from a tibialis anterior muscle. In another non-limiting example, a sample of skeletal muscle is obtained from a quadriceps muscle. Samples of muscle tissue used in a method of the present disclosure can either be fresh or have been previously stored. When previously stored, the sample may be processed prior to its use. For example, frozen muscle tissue may be washed in a suitable buffer to remove any cryoprotectant used during preservation and/or storage.

Treating a muscle tissue with an aqueous solution comprising a detergent may occur in any suitable container. A muscle sample from a donor may be provided in a container and the aqueous solution can then be added to the container. Alternatively, a container comprising the aqueous solution may be provided and a muscle sample may be placed in the container to be treated. Suitable containers are sterile or capable of being sterilized, including but not limited to circular, rectangular, or square tissue culture dishes, centrifuge tubes, flasks, or cell culture bags. Suitable containers may be formed from materials known in the art such as polymers, plastics, and glass, including polystyrene, polypropylene, and other tissue culture plastics.

Treating a muscle tissue with an aqueous solution comprising a detergent to produce a treated muscle sample typically occurs at about 15° C. to about 25° C. for at least 72 hours with agitation. Slightly elevated temperatures may be used to decrease the total treatment time or slightly lower temperatures may be used to increase the total treatment time. In some examples, treatment may occur at about 15° C. to about 25° C., about 15° C. to about 23° C., or about 15° C. to about 20° C. In some examples, treatment may occur at about 20° C. to about 30° C., about 20° C. to about 28° C., or about 20° C. to about 25° C. In some examples, treatment may occur at about 20° C. to about 30° C., about 20° C. to about 28° C., or about 20° C. to about 25° C. In some examples, treatment may occur at about 15° C. to about 25° C. for about 80 hours to about 200 hours. In some examples, treatment may occur at about 15° C. to about 25° C. for about 80 hours to about 180 hours. In some examples, treatment may occur at about 15° C. to about 25° C. for about 80 hours to about 160 hours. In other examples, treatment may occur at about 15° C. to about 25° C. for about 80 hours to about 140 hours. In certain embodiments, treatment occurs at about 15° C. to about 25° C. for about 80 hours to about 120 hours. In other embodiments, treatment occurs at about 15° C. to about 25° C. for about 100 hours to about 200 hours. In other embodiments, treatment occurs at about 15° C. to about 25° C. for about 100 hours to about 180 hours. In other embodiments, treatment occurs at about 15° C. to about 25° C. for about 100 hours to about 160 hours. In still other embodiments, treatment occurs at about 15° C. to about 25° C. for about 100 hours to about 150 hours. In still other embodiments, treatment occurs at about 15° C. to about 25° C. for about 100 hours to about 140 hours. In still other embodiments, treatment occurs at about 15° C. to about 25° C. for about 120 hours to about 150 hours. In each of the above embodiments, a method of the present disclosure may further comprise replacing the aqueous solution about every 20 hours to 30 hours, optionally about every 22 hours to 26 hours, or about every 24 hours. Agitation can be achieved by any suitable method known in the art, for example, rocking, vortexing, etc.

One or more detergents may be formulated as an aqueous solution in water (e.g., sterilized deionized water, USP water for injection, etc.). Suitable detergents may be anionic detergents including, but not limited to, alkyl sulfates and alkyl sulfonates. In one embodiment, an aqueous solution comprises about 0.5% (w/v) to about 2% (w/v) detergent in water. In another embodiment, an aqueous solution comprises about 0.5% (w/v) to about 1.5% (w/v) detergent in water. In another embodiment, an aqueous solution comprises about 0.5% (w/v) to about 1.0% (w/v) detergent in water or about 1.0% (w/v) to about 1.5% (w/v) detergent in water. In other embodiments, an aqueous solution comprises about 0.5% (w/v), about 1% (w/v), about 1.5% (w/v), or about 2% (w/v) detergent in water. As further detailed in the examples, methods of the present disclosure do not require enzymes (e.g., nucleases, proteases, carbohydrases, etc.) or chelators to be present during the treatment step. Accordingly, treating a muscle tissue with an aqueous solution comprising a detergent may occur in the absence of an enzyme and/or a chelator. In certain embodiments, an aqueous solution consists of about 0.5% (w/v) to about 2% (w/v) detergent in water. In certain embodiments, an aqueous solution consists of about 0.5% (w/v) to about 1.5% (w/v) detergent in water, about 1.0% (w/v) to about 2% (w/v) detergent in water. In certain embodiments, an aqueous solution consists of about 0.5% (w/v) to about 1.0% (w/v) detergent in water, or about 1.0% (w/v) to about 1.5% (w/v) detergent in water. In exemplary embodiments, the detergent is sodium dodecyl sulfate.

Washing the treated muscle tissue with a wash solution to produce a decellularized muscle scaffold can occur in the same container used in the treatment step. Alternatively, the treated tissue can be placed in a new, preferably sterile, container that has the same or different shape and/or size. Preferred wash solutions are sterilized, have a neutral pH, and may or may not be buffered. In an exemplary embodiment, the wash solution is sterile water. In another exemplary embodiment, the wash solution is phosphate buffered saline.

Washing typically occurs at about 15° C. to about 30° C. for about 72 hours or more with agitation. Slightly elevated temperatures may be used to decrease the total time or slightly lower temperatures may be used to increase the total time. In some examples, washing may occur at about 15° C. to about 25° C., about 15° C. to about 23° C., or about 15° C. to about 20° C. In some examples, washing may occur at about 20° C. to about 30° C., about 20° C. to about 28° C., or about 20° C. to about 25° C. In some embodiments, washing occurs at about 15° C. to about 25° C. for about 72 hours to about 120 hours. In other embodiments, washing occurs at about 15° C. to about 25° C. for about 84 hours to about 120 hours. In other embodiments, washing occurs at about 15° C. to about 25° C. for about 96 hours to about 120 hours. In other embodiments, washing occurs at about 15° C. to about 25° C. for about 72 hours to about 108 hours. In still other embodiments, washing occurs at about 15° C. to about 25° C. for about 72 hours to about 96 hours. During the washing, the wash solution is replaced at substantially even intervals. In some embodiments, the wash solution is replaced at least 4 times, preferably at least 5 times, more preferably at least 6 times. Agitation can be achieved by any suitable method known in the art, for example, rocking, vortexing, etc.

In exemplary embodiment, a method of the present disclosure comprises: (a) providing a muscle sample from a mammal, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a mammal, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a rodent, optionally a rat, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a rodent, optionally a rat, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a canine, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a canine, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a feline, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a feline, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a horse, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a horse, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a sheep, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a sheep, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a human, and treating the muscle sample with an aqueous solution comprising about 0.5% (w/v) to about 2% (w/v) of an anionic detergent or about 1% (w/v) to about 2% (w/v) of an anionic detergent for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

In exemplary embodiment, a method of the present disclosure comprises: (a) providing a muscle sample from a human, and treating the muscle sample with an aqueous solution comprising about 0.5% (w/v) to about 2% (w/v) sodium dodecyl sulfate or about 1% (w/v) to about 2% (w/v) sodium dodecyl sulfate for greater than 72 hours at about 15° C. to about 25° C., with agitation, to produce a treated muscle sample, wherein the aqueous solution is replaced about every 20 to 28 hours; (b) removing the aqueous solution and adding a wash solution to the treated muscle sample, and then agitating for about 72 hours or more at about 15° C. to about 25° C. (“wash period”) to produce a decellularized muscle scaffold, wherein the wash solution is replaced at least four times during the wash period, preferably five or six times. In certain embodiments, the muscle sample is treated for about 80 hours to about 200 hours, optionally about 120 hours to about 200 hours or about 140 hours to about 200 hours.

Following the wash period, a method of the present disclosure may further comprise storage of the decellularized muscle scaffold in a suitable storage buffer. A non-limiting example of a suitable storage buffer is PBS. Suitable storage buffers preferably have a neutral pH and are sterilized, and may optionally include one or more antimicrobial agent, preservative, or cryoprotectant. In some embodiments, a decellularized muscle scaffold is stored at about 4° C. In other embodiments, a decellularized muscle scaffold is stored at about 0° C. or lower.

Following the wash period, a method of the present disclosure may further comprise freezing the decellularized muscle scaffold. Alternatively, the method of the present disclosure may further comprise lyophilizing (freeze drying) the decellularized muscle scaffold.

### II. Decellularized Muscle Scaffolds

In another aspect, the present disclosure provides decellularized muscle scaffold produced by a method of Section I.

In another aspect, the present disclosure provides a decellularized muscle scaffold produced by a method of Section I that is treated to attract endogenous muscle stem cells when transplanted in a subject. As used herein, the term “muscle stem cell” refers to any cell capable of giving rise to a fully differentiated muscle cell, including a smooth muscle cell, a skeletal muscle cell, or a cardiac muscle cell. Non-limiting examples of muscle stem cells include embryonic stem cells, induced-pluripotent stem cells, mesenchymal stem cells, cardiac stem cells, muscle-derived stem cells, muscle progenitor cells, mesodermal precursor cells, mesenchymal stromal cells, myoblasts, mesoangioblasts, muscular tissue pericytes, satellite cells, fibroadipogenic progenitors, Pax3⁺, Sk-34, CD45⁺/Sca1⁺ and PW1⁺/Pax7⁻ interstitial cells, skeletal muscle derived stem cells, muscle side population cells, etc. Treating a decellularized muscle scaffold to attract endogenous muscle stem cells when implanted in a subject generally comprises impregnating the decellularized muscle scaffold with one or more factors (e.g., proteins, cell types, cytokines, growth factors, transcription factors, etc.) that promote the recruitment of one or more muscle stem cell type to the scaffold following transplantation of the scaffold into a subject.

In another aspect, the present disclosure provides a decellularized muscle scaffold produced by a method of Section I that is seeded with a plurality of muscle stem cells. A “plurality of muscle stem cells” refers to one or more than one type of muscle stem cell in amounts ranging from about 1×10² cells to about 1×10²⁰ cells per 3 cm², or from about 1×10⁵ cells to about 1×10¹⁰ cells per 3 cm² or even about 1×10⁵ cells to about 1×10¹⁰ cells per 3 cm². Methods for isolating muscle stem cells, culturing muscle stem cells, and re-seeding tissue scaffolds are known in the art and further detailed in the examples. The decellularized muscle scaffold seeded with a plurality of muscle stem cells may further comprise one or more factors that promote the recruitment of one or more muscle stem cell type to the scaffold following transplantation of the scaffold into a subject, and/or one or more factors that promote the differentiation of the implanted muscle stem cell(s) following transplantation of the scaffold into a subject.

### III. Use of Decellularized Muscle Scaffolds

In another aspect, the present disclosure provides for use of decellularized muscle scaffolds of Section II to treat a subject with damaged or lost muscle. Generally, the method comprises transplanting a decellularized muscle scaffold of Section II into an affected area in a subject in need thereof. Decellularized muscle scaffolds offer a preformed, native ECM, which can be either preseeded with muscle stem cells or treated to attract endogenous muscle stem cells, as described in Section II. In some embodiments, the damaged or lost muscle is skeletal muscle. In other embodiments, the damaged or lost muscle is smooth muscle. In still other embodiments, the damaged or lost muscle is cardiac muscle. Muscle scaffolds can be selected so that the native microenvironment and mechanical properties are similar to the tissue being repaired, allowing for optimal muscle stem cell adhesion and migration, which are essential in myogenesis. Suitable muscle scaffolds may be produced from a donor muscle tissue of the same species as the subject in need of treatment, or from a different species as the subject in need of treatment. In some embodiments, the decellularized muscle scaffold is produced from a muscle sample from a donor that is the same muscle type as the damaged or lost muscle in the subject. In further embodiments, the decellularized muscle scaffold is seeded with muscle stem cells that are capable of producing a muscle cell type comprising the damaged or lost muscle in the subject. In other embodiments, the decellularized muscle scaffold is produced from a muscle sample from a donor that is a different muscle type as the damaged or lost muscle in the subject. In further embodiments, the decellularized muscle scaffold is seeded with muscle stem cells that are capable of producing a muscle cell type comprising the damaged or lost muscle in the subject. Decellularization muscle scaffolds of the present disclosure have conserved architectural features of the tissue (e.g., the vascular bed, etc.). As such, when implanted, decellularized muscle scaffolds of the present disclosure integrate readily with endogenous tissue, and demonstrate early signs of integration, myocyte regeneration, fibrosis, and/or neoangiogenesis. Measures of integration, myocyte regeneration, fibrosis, and neoangiogenesis are known in the art, and further detailed in the examples.

Treatment can result in an increase in muscle mass, muscle function, or both. In preferred embodiments, treated subjects have improved muscle mass and improved muscle function. For example, muscle function may improve by about 10% to about 50% (e.g., inclusive of about 10%, about 20%, about 30%, about 40% and about 50%) as compared to pre-treatment. Alternatively, muscle function may improve by about 60% to about 100% (e.g., inclusive of about 60%, about 70%, about 80%, about 90%, and about 100%) as compared to pre-treatment. In another example, muscle function may improve by about 100% to about 500% or more as compared to pre-treatment. Methods for evaluating muscle function are known in the art and include, but are not limited to, measures of voluntary or electrically-evoked isometric strength, dynamic strength, fatigability, contraction, relaxation, etc. Use of peak isometric tetanic force is detailed in the examples.

The subject can be selected from a mammal, bird, fish, reptile, and amphibian. A subject that is a mammal can be a human or a non-human mammal, such as a livestock animal, a companion animal, a lab animal, a zoological animal, etc. In one embodiment, a donor may be a rodent, e.g., a mouse, a rat, a guinea pig, etc. In another embodiment, a donor may be a livestock animal. Non-limiting examples of suitable livestock animals include pigs, cows, horses, goats, sheep, llamas and alpacas. In yet another embodiment, a donor may be a companion animal. Non-limiting examples of companion animals include pets such as dogs, cats, rabbits, etc. In yet another embodiment, a donor may be a zoological animal. As used herein, a “zoological animal” refers to an animal that may be found in a zoo. Such animals include non-human primates, large cats, wolves, bears, etc. In certain embodiments, the animal is a laboratory animal. Non-limiting examples of a laboratory animal include rodents, rabbits, canines, felines, non-human primates, etc.

In one embodiment, decellularized muscle scaffolds of Section I that are seeded with a plurality of muscle stem cells, as described in of Section II, are used to replace damaged or lost skeletal muscle in a mammal. The mammal may have damaged or lost skeletal muscle due to blunt trauma, sharp trauma, chronic demyelination and/or denervation (e.g., due to Multiple sclerosis, Alzheimer's disease, Parkinson's disease and PD-related disorders, prion disease, Huntington's disease, spinocerebellar ataxia, spinal muscular atrophy, or any other neurodegenerative disease), tumor extirpation, muscle degeneration (e.g., due to muscular dystrophy, or any other disease or disorder) etc. In certain embodiments, the mammal may have volumetric muscle loss (VML). Skeletal muscle tissue used in the scaffold may be obtained from any mammal donor, preferably from a donor of the same species, and seeded with embryonic stem cells, induced-pluripotent stem cells, muscle-derived stem cells, muscle progenitor cells, mesodermal precursor cells, mesenchymal stromal cells, mesoangioblasts, muscular tissue pericytes, satellite cells, fibroadipogenic progenitors, Pax3⁺, Sk-34, CD45⁺/Sca1⁺ and PW1⁺/Pax7⁻ interstitial cells, skeletal muscle derived stem cells, muscle side population cells, or any combination thereof. In some embodiments, the decellularized muscle scaffold was produced from skeletal muscle tissue obtained from a mammal of the same species, optionally a sample of skeletal muscle obtained from a tibialis anterior muscle, and then seeded with a plurality of mesenchymal stromal cells, mesoangioblasts, muscular tissue pericytes, satellite cells, skeletal muscle derived stem cells, muscle side population cells, or any combination thereof. In some embodiments, the decellularized muscle scaffold was produced from skeletal muscle tissue obtained from a mammal of the same species, optionally a sample of skeletal muscle obtained from a tibialis anterior muscle, and then seeded with a plurality of mesenchymal stromal cells. In some embodiments, the decellularized muscle scaffold was produced from skeletal muscle tissue obtained from a mammal of the same species, optionally a sample of skeletal muscle obtained from a tibialis anterior muscle, and then seeded with a plurality of mesoangioblasts and/or muscular tissue pericytes. In some embodiments, the decellularized muscle scaffold was produced from skeletal muscle tissue obtained from a mammal of the same species, optionally a sample of skeletal muscle obtained from a tibialis anterior muscle, and then seeded with a plurality of skeletal muscle derived stem cells and/or muscle side population cells. In some embodiments, the decellularized muscle scaffold was produced from skeletal muscle tissue obtained from a mammal of the same species, optionally a sample of skeletal muscle obtained from a tibialis anterior muscle, and then seeded with a plurality of satellite cells. In certain of the above embodiments, the muscle stem cell is derived from a mammal of the same species, optionally from the mammal in need of treatment.

In one embodiment, decellularized muscle scaffolds of Section I that are seeded with a plurality of muscle stem cells, as described in of Section II, are used to replace damaged or lost skeletal muscle in a human. The human may have damaged or lost skeletal muscle due to blunt trauma, sharp trauma, chronic demyelination and/or denervation (e.g., due to Multiple sclerosis, Alzheimer's disease, Parkinson's disease and PD-related disorders, prion disease, Huntington's disease, spinocerebellar ataxia, spinal muscular atrophy, or any other neurodegenerative disease), tumor extirpation, muscle degeneration (e.g., due to muscular dystrophy, or any other disease or disorder) etc. In certain embodiments, the human may have volumetric muscle loss (VML). Skeletal muscle tissue used in the scaffold may be obtained from any human or non-human mammal donor, preferably from a human donor, and seeded with embryonic stem cells, induced-pluripotent stem cells, muscle-derived stem cells, muscle progenitor cells, mesodermal precursor cells, mesenchymal stromal cells, mesoangioblasts, muscular tissue pericytes, satellite cells, fibroadipogenic progenitors, Pax3⁺, Sk-34, CD45⁺/Sca1⁺ and PW1⁺/Pax7⁻ interstitial cells, skeletal muscle derived stem cells, muscle side population cells, or any combination thereof. In some embodiments, the decellularized muscle scaffold was produced from skeletal muscle tissue obtained from a human, optionally a sample of skeletal muscle obtained from a tibialis anterior muscle, and then seeded with a plurality of mesenchymal stromal cells, mesoangioblasts, muscular tissue pericytes, satellite cells, skeletal muscle derived stem cells, muscle side population cells or any combination thereof. In some embodiments, the decellularized muscle scaffold was produced from skeletal muscle tissue obtained from a human, optionally a sample of skeletal muscle obtained from a tibialis anterior muscle, and then seeded with a plurality of mesenchymal stromal cells. In some embodiments, the decellularized muscle scaffold was produced from skeletal muscle tissue obtained from a human, optionally a sample of skeletal muscle obtained from a tibialis anterior muscle, and then seeded with a plurality of mesoangioblasts and/or muscular tissue pericytes. In some embodiments, the decellularized muscle scaffold was produced from skeletal muscle tissue obtained from a human, optionally a sample of skeletal muscle obtained from a tibialis anterior muscle, and then seeded with a plurality of skeletal muscle derived stem cells and/or muscle side population cells. In some embodiments, the decellularized muscle scaffold was produced from skeletal muscle tissue obtained from a human, optionally a sample of skeletal muscle obtained from a tibialis anterior muscle, and then seeded with a plurality of satellite cells. In certain of the above embodiments, the muscle stem cell is derived from a human, optionally from the human in need of treatment.

In one embodiment, decellularized muscle scaffolds of Section I that are seeded with a plurality of muscle stem cells, as described in of Section II, are used to replace damaged or lost skeletal muscle in a livestock animal. The livestock animal may have damaged or lost skeletal muscle due to blunt trauma, sharp trauma, chronic demyelination and/or denervation, tumor extirpation, muscle degeneration, etc. Skeletal muscle tissue used in the scaffold may be obtained from any human or non-human mammal donor, preferably from a donor that is same species as the animal in need of treatment, and seeded with embryonic stem cells, induced-pluripotent stem cells, muscle-derived stem cells, muscle progenitor cells, mesodermal precursor cells, mesenchymal stromal cells, mesoangioblasts, muscular tissue pericytes, satellite cells, fibroadipogenic progenitors, Pax3⁺, Sk-34, CD45⁺/Sca1⁺ and PW1⁺/Pax7⁻ interstitial cells, skeletal muscle derived stem cells, muscle side population cells, or any combination thereof. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesenchymal stromal cells, mesoangioblasts, muscular tissue pericytes, satellite cells, skeletal muscle derived stem cells, muscle side population cells, or any combination thereof. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesenchymal stromal cells. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesoangioblasts and/or muscular tissue pericytes. In some embodiments, the decellularized muscle scaffold was produced from skeletal muscle tissue obtained from a donor that is of the same species, optionally a sample of skeletal muscle obtained from a tibialis anterior muscle, and then seeded with a plurality of skeletal muscle derived stem cells and/or muscle side population cells. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of satellite cells. In certain of the above embodiments, the muscle stem cell is derived from a donor that is same species as the animal in need of treatment, optionally from the animal in need of treatment.

In one embodiment, decellularized muscle scaffolds of Section I that are seeded with a plurality of muscle stem cells, as described in of Section II, are used to replace damaged or lost skeletal muscle in a companion animal. The companion animal may have damaged or lost skeletal muscle due to blunt trauma, sharp trauma, chronic demyelination and/or denervation, tumor extirpation, muscle degeneration, etc. Skeletal muscle tissue used in the scaffold may be obtained from any human or non-human mammal donor, preferably from a donor that is same species as the animal in need of treatment, and seeded with embryonic stem cells, induced-pluripotent stem cells, muscle-derived stem cells, muscle progenitor cells, mesodermal precursor cells, mesenchymal stromal cells, mesoangioblasts, muscular tissue pericytes, satellite cells, fibroadipogenic progenitors, Pax3⁺, Sk-34, CD45⁺/Sca1⁺ and PW1⁺/Pax7⁻ interstitial cells, skeletal muscle derived stem cells, muscle side population cells, or any combination thereof. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesenchymal stromal cells, mesoangioblasts, muscular tissue pericytes, satellite cells, skeletal muscle derived stem cells, muscle side population cells, or any combination thereof. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesenchymal stromal cells. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesoangioblasts and/or muscular tissue pericytes. In some embodiments, the decellularized muscle scaffold was produced from skeletal muscle tissue obtained from a donor that is of the same species, optionally a sample of skeletal muscle obtained from a tibialis anterior muscle, and then seeded with a plurality of skeletal muscle derived stem cells and/or muscle side population cells. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of satellite cells. In certain of the above embodiments, the muscle stem cell is derived from a donor that is same species as the animal in need of treatment, optionally from the animal in need of treatment.

In one embodiment, decellularized muscle scaffolds of Section I that are seeded with a plurality of muscle stem cells, as described in of Section II, are used to replace damaged or lost skeletal muscle in a laboratory animal. The laboratory animal may have damaged or lost skeletal muscle due to blunt trauma, sharp trauma, chronic demyelination and/or denervation, tumor extirpation, muscle degeneration, etc. Skeletal muscle tissue used in the scaffold may be obtained from any human or non-human mammal donor, preferably from a donor that is same species as the animal in need of treatment, and seeded with embryonic stem cells, induced-pluripotent stem cells, muscle-derived stem cells, muscle progenitor cells, mesodermal precursor cells, mesenchymal stromal cells, mesoangioblasts, muscular tissue pericytes, satellite cells, fibroadipogenic progenitors, Pax3⁺, Sk-34, CD45⁺/Sca1⁺ and PW1⁺/Pax7⁻ interstitial cells, skeletal muscle derived stem cells, muscle side population cells, or any combination thereof. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesenchymal stromal cells, mesoangioblasts, muscular tissue pericytes, satellite cells, skeletal muscle derived stem cells, muscle side population cells, or any combination thereof. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesenchymal stromal cells. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of mesoangioblasts and/or muscular tissue pericytes. In some embodiments, the decellularized muscle scaffold was produced from skeletal muscle tissue obtained from a donor that is of the same species, optionally a sample of skeletal muscle obtained from a tibialis anterior muscle, and then seeded with a plurality of skeletal muscle derived stem cells and/or muscle side population cells. In some embodiments, the decellularized muscle scaffold was produced from skeletal muscle tissue obtained from a donor that is same species as the animal in need of treatment, optionally a sample of skeletal muscle obtained from a tibialis anterior muscle, and then seeded with a plurality of satellite cells. In certain of the above embodiments, the muscle stem cell is derived from a donor that is same species as the animal in need of treatment, optionally from the animal in need of treatment.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### EXAMPLES

The following examples illustrate various iterations of the invention.

### Example 1

Tumor surgery often requires removal of a large margin of normal and affected muscle tissue in order to ensure complete removal, particularly with sarcomas. The muscle loss resulting from such surgery can be partially filled by autologous tissue transfer of muscle from other areas of the body, however the functional and cosmetic outcomes using such techniques are often less than ideal.¹ Donor site morbidity is high, and function at the site of reconstruction is commonly poor.²⁻⁴ In response to this challenging clinical problem, a tissue engineering approach involving regeneration and re-animation of muscle in the area of muscle loss is highly intriguing solution.

The current paradigm for tissue engineering strategies involves the combination of three essential items: scaffolds, cells, and signal. Scaffolds can be biologic or manufactured. Endogenous or exogenous cells provide the regenerative and healing capacity. Signal can be introduced in a deliberate way using recombinant proteins or, if the scaffold is of biological origin, it may contain the salient biological signals on its own. These signals guide nearby repair cells to behave in a certain way. One example of this phenomenon is the use of bone scaffold. The acellular bone scaffold contains BMP-2, a potent osteogenic protein as well as a topographical environment ideal for osteocyte adherence and growth. When combined with bone marrow aspirate (exogenous cells) or implanted in a fracture site (endogenous cells), the multi-potent stem cells respond to the BMP-2 by differentiating into osteoblasts. The combination of the topography of the scaffold material and the differentiated cells contributes to a more rapid healing response.

While bone scaffolds have been well studied, soft tissue scaffolds represent an emerging area in tissue engineering. In particular, decellularized soft tissue scaffolds are an exciting novel component of novel tissue, such as engineered organ and tissue products. Composed of natural extracellular matrix, they have been shown to be remarkably biocompatible and show great promise for tissue regeneration. Recently, investigators have shown that decellularized scaffolds, in particular skeletal muscle, promote anti-inflammatory and immunosuppressive responses both in vitro and in vivo—two important features that protect against tissue rejection and allow for the possibility of using donated tissue that can be stored for long periods of time. To-date, clinical applications have been limited to static tissues such as skin. New evidence now suggests that undifferentiated mesenchymal stem cells will not only successfully survive on such decellularized tissue scaffolds when seeded there, but that the signals provided by the scaffolds can guide differentiation of these multi-potent cells into the original mesenchymal tissue of origin. The most recent example is the successful differentiation of functioning myocardiocytes from pluripotent fibroblasts on decellularized heart muscle in vitro.⁵

Described here is an approach wherein decellularized allograft muscle scaffold is “re-animated” using muscle stem cells and implanted into an area of massive muscle loss to create a functional muscle. It is shown that muscle stem cells can be successfully seeded onto a decellularized skeletal muscle scaffold of the present disclosure and implanted into muscle defects wherein the muscle stem cells will differentiate and proliferate in vivo. It is also shown that this allogeneic tissue construct will integrate into recipient tissue with minimal inflammation and functions with greater contractility than muscles that have either not been reconstructed or those reconstructed with scaffold alone.

Decellularized Muscle Scaffold:

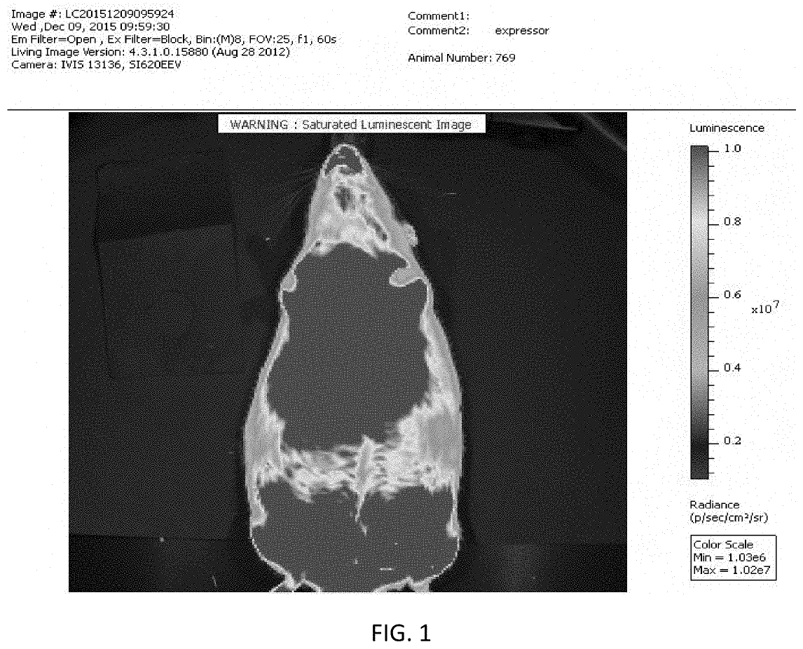

All procedures were conducted in compliance with the Animal Welfare Act and were approved by the Institutional Animal Care and Use Committee at Colorado State University. Transgenic universally expressing Luciferase Wistar rats (FIG. 1) weighing 250-350 grams were used for muscle tissue donors. The tibialis anterior (TA) muscle was removed bilaterally and decellularized as described below. Decellularization was confirmed using bioluminescent imaging (BLI) with the IVIS® Spectrum, scanning electron microscopy, confocal microscopy, and DNA analysis using DNAeasy (Qiagen Technologies).

Muscle scaffolds measuring approximately 3 cm L×1 cm W×1 cm H were decellularized as follows. The tibialis anterior muscle was dissected using sterile technique, and placed in a sterile 50 ml centrifuge tube containing sterile 1% Sodium Dodecyl Sulfate (SDS) in Deionized water. The tube was then placed on a vortex mixer set to 5-7 and allowed to shake at room temperature for 120 hours. During that time the SDS was aspirated and replaced using sterile technique once every 24 hours. Following the 120 hr in SDS, the muscle tissue remained within the sterile centrifuge tube in the vortex mixer at room temperature and the SDS was aspirated and replaced with sterile phosphate buffered saline. A rinse cycle was initiated. This involved PBS being aspirated and replaced a minimum of 6 times during this 3-5 day period of time. Scaffolds were then stored at 4° C. in PBS.

Following this process, treated muscles were analyzed for decellularization by SEM using previously described techniques using SEM and DNA analysis. Briefly, the scaffolds were fixed in SEM fixative (3% Gluteraldehyde in 0.1 M sodium cacodylate+0.1 M sucrose) for 45 minutes and placed in SEM buffer (0.34 g sucrose+0.21 g sodium cacodylate in 10 ml DI water) for 10 minutes. The samples were then dehydrated by placing in gradations of ethanol for 10 minutes each (35%, 50%, 70%. 100%) then overnight in HMDS (hexamethyldisilazine) with the lid loose to dry off completely. Muscle was lyophilized and mounted onto aluminum stubs using copper conductive tape and colloidal graphite and allowed to dry overnight. A 10 nm gold coating was sputtered coated onto the tissue and then imaged using a JEOL JSM-6500F scanning electron microscope (Tokyo, Japan) with an accelerating voltage of 15 kV.

Decellularized scaffolds were analyzed for DNA using a Qiagen DNAeasy Blood and Tissue Kit. Scaffolds as well as normal muscle (control) were prepped and samples were thawed. Samples up to 25 mg in weight were placed into microcentrifuge tubes. To each tube, 180 μl Buffer ATL and 20 μl proteinase K was added and samples were sonicated at 56° C. for 1-3 hours until completely lysed. Buffer AL (200 μl) was added to each, vortexed and placed back in 56° C. for 10 minutes. Ethanol was added at 200 μl, mixed and the mixture was placed in DNAeasy Mini spin column in a 2 ml collection tube. Samples were centrifuged at 8000 rpm for 1 min, after which the column was placed in new 2 ml collection tube. To the column, 500 μl AW1 buffer was added and the sample was centrifuged for 1 minute at 8000 rpm. The column was then placed in a new 2 ml tube and 500 μl AW2 was added and the sample was centrifuged for 3 minutes at 14,000 rpm. The column was put into a final 2 ml microcentrifuge tube and the DNA was eluted by adding 200 μl Buffer AE. DNA content was read using a Nanodrop spectrophotometer.

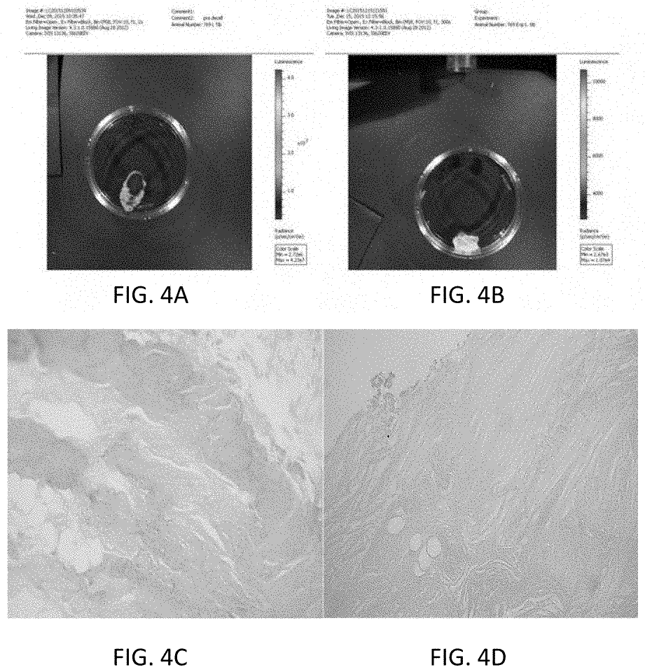

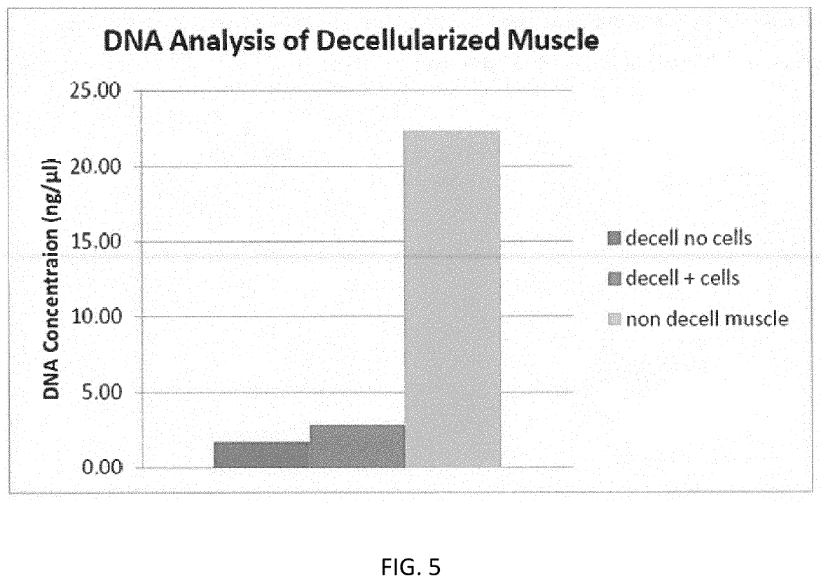

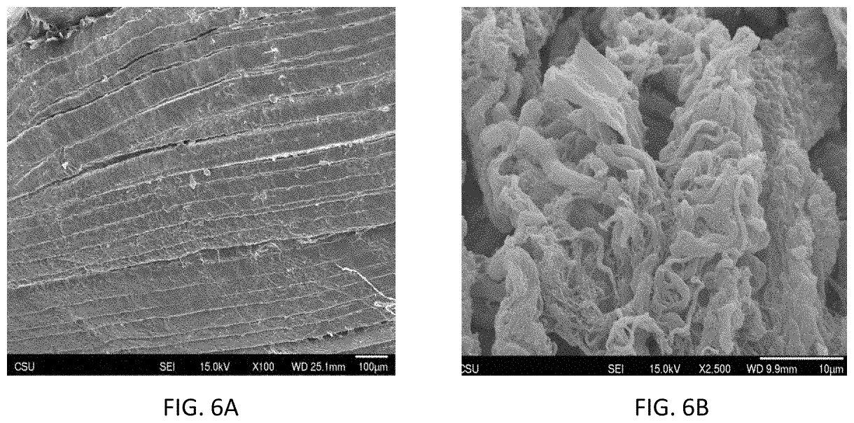

Decellularization of the TA muscle was successfully performed and validated with the absence of luciferase expression on bioluminescent imaging (FIG. 4), absence of nuclei on H&E confocal microscopy (FIG. 4), and minimal DNA identified on DNA analysis of the scaffold (FIG. 5). SEM was also performed and indicative that the decellularized scaffold retained its architecture (FIG. 6).

Re-Animated Muscle Scaffold:

Transgenic universally expressing luciferase rats were used as skeletal muscle mesenchymal stromal cell donors. Briefly, quadriceps muscle was minced, rinsed with DMEM (low glucose) containing Antibiotic Antimycotic and added to a freshly prepared collagenase solution (1 mg/ml DMEM low glucose) and stirred at 37° C. for 45 minutes. The solution was placed in a conical tube, with DMEM (low glucose) containing antibiotics and antimycotics. The mixture was centrifuged for 5 minutes at 2000 rpm, resuspended in same supernatant and tube, and centrifuged a second time for 5 minutes at 2000 rpm. The supernatant was then removed and the pellet re-suspended in DMEM Low glucose containing 15% FBS, MEM vitamins, Nonessential amino acids, and antibiotic/antimycotic (MSC Media) and plated into a 150 cm flask. The flask was allowed to incubate undisturbed for 4 days. The cells were fed at this time and every three days with fresh MSC media until confluent and then passaged routinely. Passages 3-6 were used for all experiments. The cells were imaged with the IVIS® Spectrum to confirm luciferase expression.

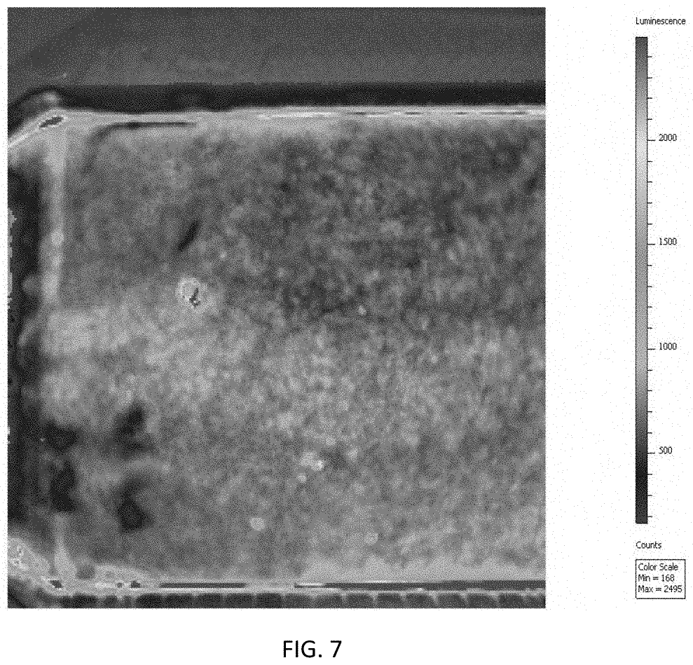

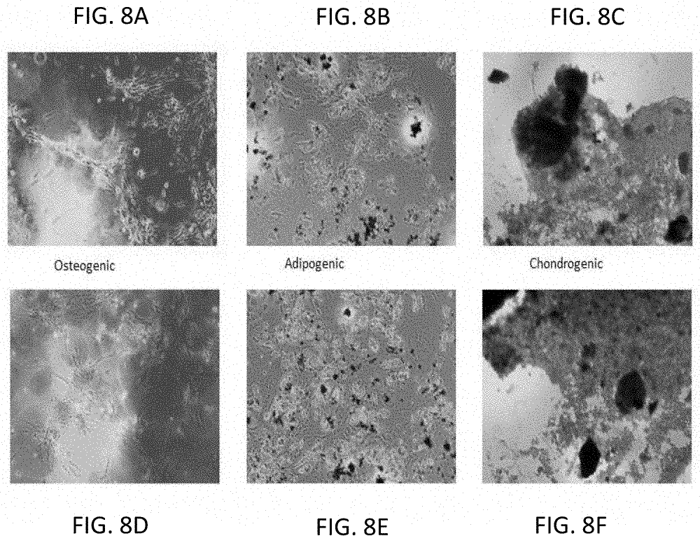

The Luc+SMdMSCs were successfully harvested from Wistar quadriceps muscle and culture expanded (FIG. 7). Trilineage differentiation was performed to validate multipotency of the harvested cells (FIG. 8).

Seventy-two hours prior to surgical implantation, each decellularized muscle scaffold (DMS) was seeded with 1×10⁶ Luc+SMdMSCs. Immediately before implantation, media was rinsed and successful adherence of the Luc+SMdMSCs onto the muscle scaffold was evaluated using the IVIS® Spectrum. During development of the re-animated muscle scaffold, seeded scaffold was formalin fixed and paraffin embedded for routine H&E staining and histologic evaluation 72 hours after seeding. All DMS scaffolds, with and without cells were treated with FGF (5 ng/ml) in the media 72 hours prior to implantation.

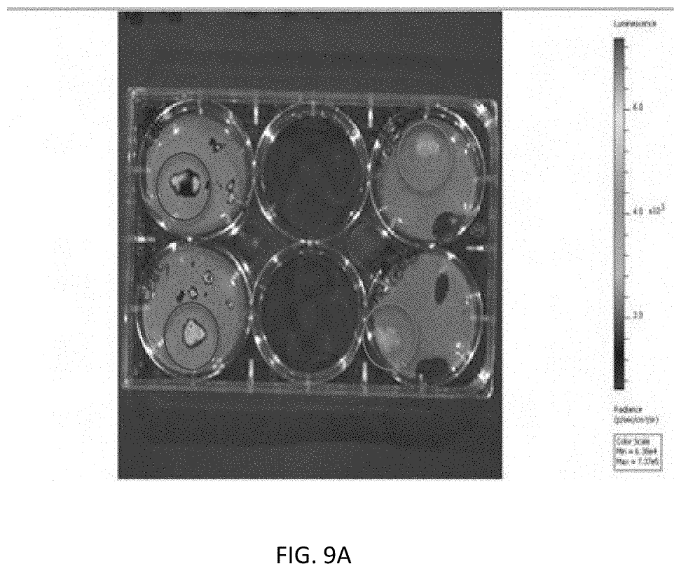

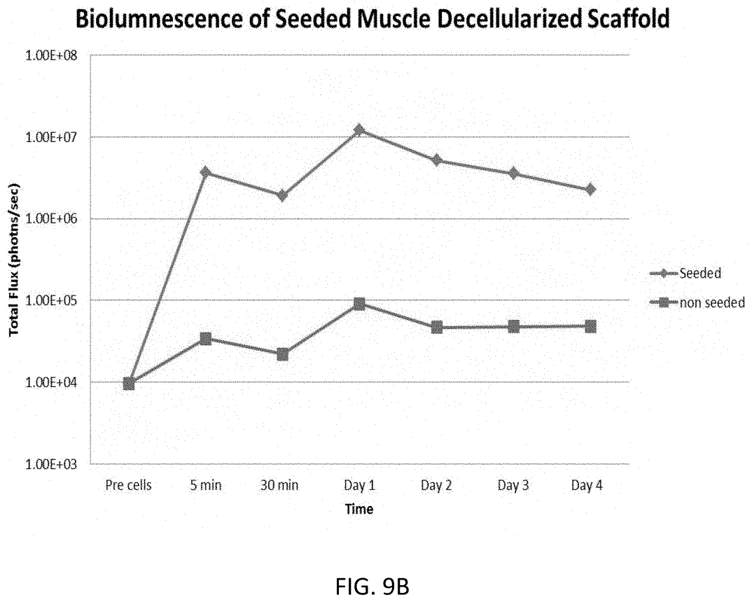

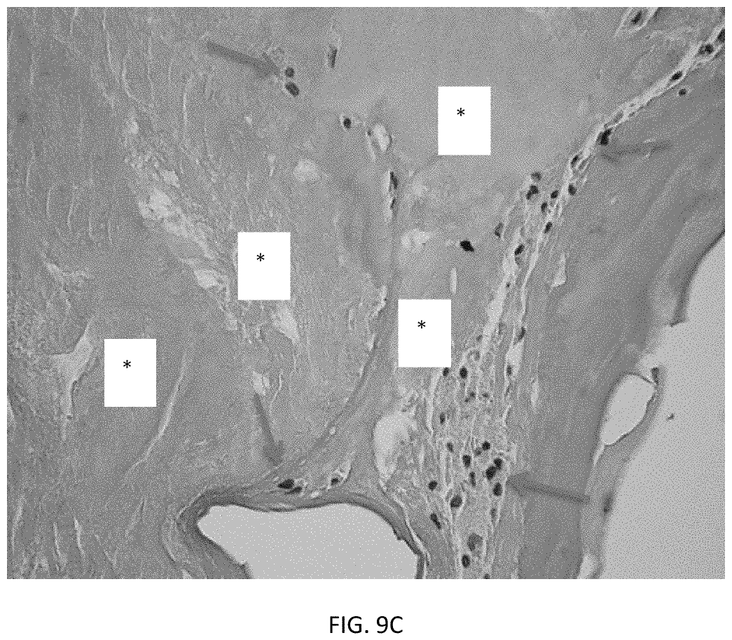

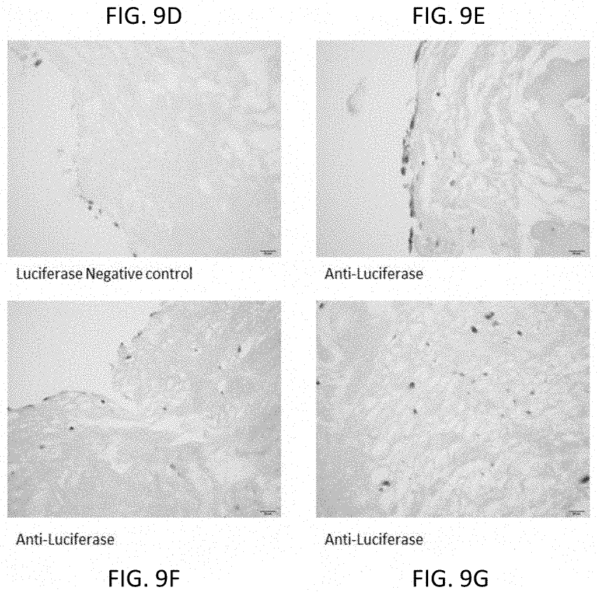

The Luc+SMdMSCs were successfully seeded onto the DMS, remained adherent to the DMS, and luciferase expression did not diminish for 96 hours in culture as visualized on BLI (FIGS. 9A and 9B). Evaluation of H&E staining and IHC for Luciferase also confirmed adherence of the cells onto the scaffold (FIGS. 9C and 9D). The cells were predominantly visualized on the superficial layers of the scaffold.

Surgical Procedure:

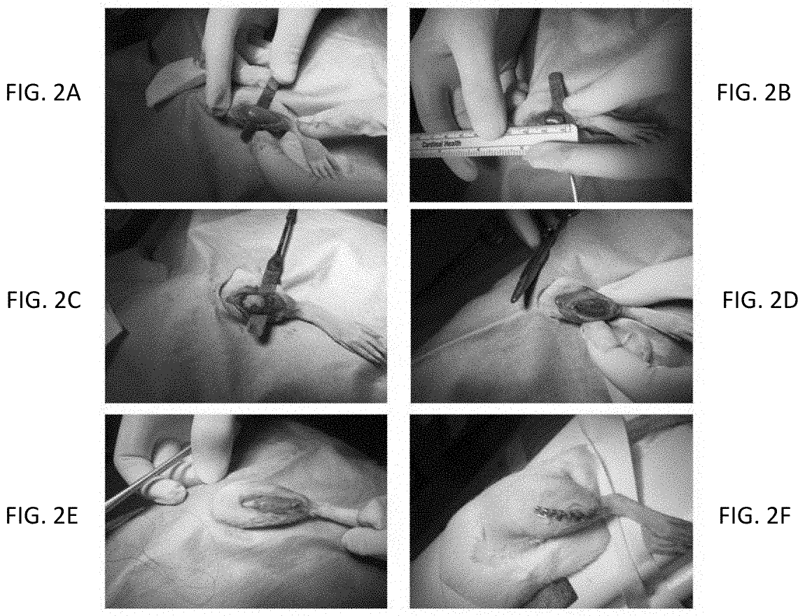

Twenty-seven Wistar rats weighing 300-350 grams were used as recipients. A well-characterized model of volumetric muscle loss was used for the study (FIG. 2).⁶ This model utilizes a critical-sized muscle defect that will not spontaneously heal and consistently produces peak isometric tetanic force loss of 32% at 4 months post-operative compared to un-operated muscles. Briefly, under general anesthesia and sterile surgical conditions, a 10 mm×7 mm×3 mm defect was made using sharp dissection in the midbody of the TA muscle. The distal tendon and proximal insertion was left intact. Immediately after creation of the defect, the scaffold was sutured to the remaining TA muscle with 6-0 Prolene taking care to include the epimysium of the TA muscle in the suture to improve tension—holding power. Empty defects remained empty, however 6-0 Prolene was placed at the four corners of the defect for easier identification of the defect area during histologic evaluation. Subcutaneous tissue and skin were closed routinely

Animals were given long-acting buprenorphine subcutaneously (1.0 mg/kg) prior to surgery and then as needed afterwards based upon observed pain and lameness scales established in the investigator's laboratory. The treatment groups consisted of DMS/Luc+SMdMSCs (n=9), DMS only (n=9), and empty defect (n=9). An a priori power analysis suggested that n=8 would provide a power of 0.8 to detect a difference of 35% in peak isometric tetanic force between groups. Nine animals were included per group to allow for possible attrition.

All rats survived surgery and no appreciable lameness or infection was observed in any animal. There were no complications with wound healing.

In Vivo Imaging:

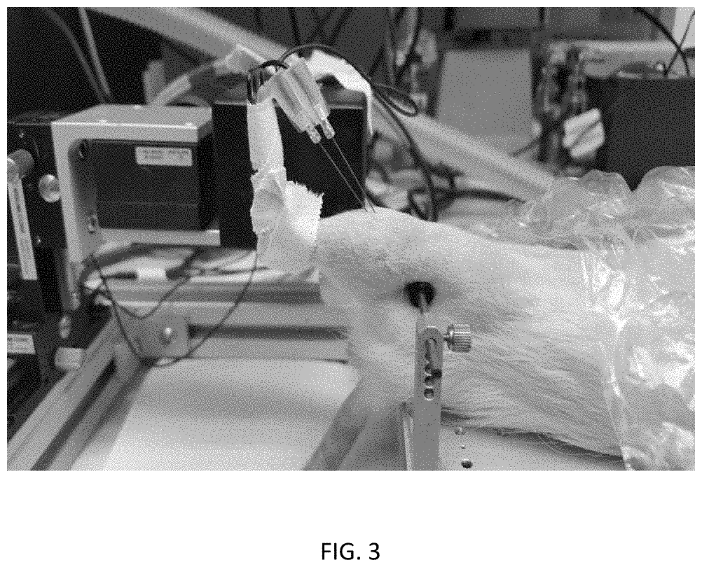

Two days after surgery, all rats were imaged in the IVIS® Spectrum to assess luciferase expression at the surgery site (FIG. 3). After this initial evaluation, once weekly imaging was performed only in the rats that received a repair with DMS and Luc+SMdMSCs.

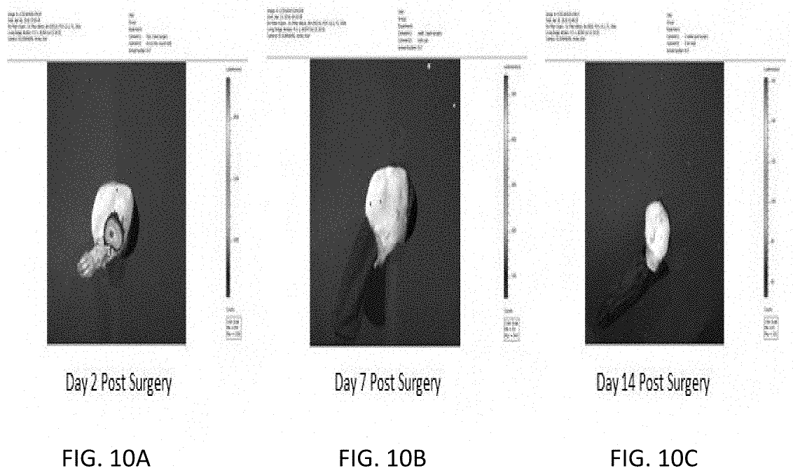

Luciferase expression was present at the surgery site in all animals that received Luc+SMdMSCs immediately following surgery. This expression diminished to an undetectable level by two weeks following surgery (FIG. 10).

In Vivo Peak Isometric Tetanic Force Measurement:

At 4 months post-implantation, peak isometric tetanic force (P₀) was measured as previously described in both the operated and un-operated contralateral limbs using an Aurora Scientific Muscle Lever Testing System.⁶ Briefly, each rat was placed under general anesthesia with 2-4% Isoflurane in 100% Oxygen. The hind limbs were clipped of all fur. The achilles tendon and extensor digitalis longus (EDL) were severed bilaterally prior to P₀ testing. The rat was placed in the testing jig on top of a circulating warm water bed. The ankle and stifle joints were placed at 90° to each other and the foot and ankle were secured into the lever system with tape. The stifle joint was positioned using stabilization pins (FIG. 3). Two electrodes were placed under skin and touching the TA fascia, and the optimal frequency for tetanic contraction was identified (from 100-200 Hz). P₀ was then measured two additional times at the optimal frequency with a two minute rest period between contractions. Investigators were blinded to treatment group during measurement of P₀. Results were then compared between the operated and un-operated contralateral controls and then between groups. All rats were sacrificed following functional muscle testing and the operated muscle was harvested.

One rat in the DMS/Luc+SMdMSCs group died during P₀ testing and therefore those results were censored. P₀ testing for the un-operated control leg could not be optimized for one rat in the empty defect group and therefore those results were also censored.

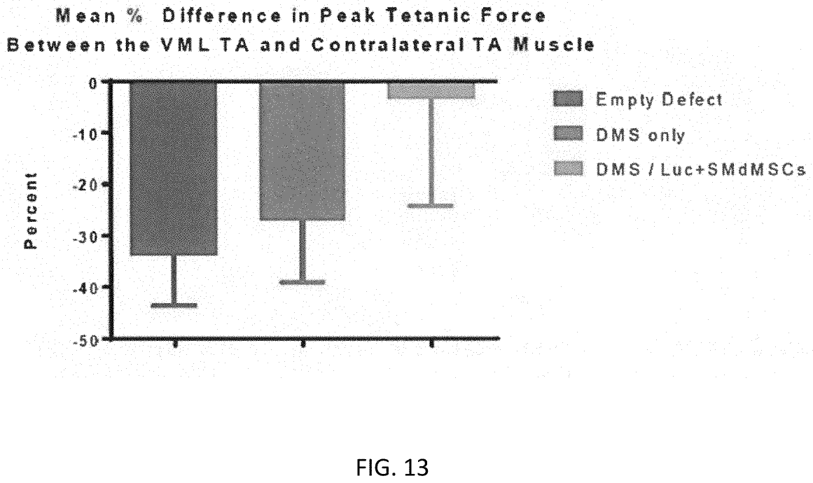

Empty defect rats recovered on average 66% of normal P₀ at 4 months, DMS scaffold rats recovered 73% of normal P₀ and Luc+SMdMSCs treated rats recovered 97% of normal P₀. A statistical difference between groups was not achieved (p>0.05; FIG. 13).

Ex Vivo and Histologic Measures:

Body weight at the time of euthanasia was recorded. Operated and un-operated contralateral control TA muscles were harvested for whole muscle fluorescence imaging, muscle weight and histological evaluation. The EDL muscles were also weighed. Sections were processed for standard H&E and Masons Trichrome staining to permit evaluation of integration, inflammation, myocyte response, and fibrosis scoring. Inflammation, integration, myocyte response, and fibrosis scoring and descriptions were performed by a board certified veterinary pathologist blinded to treatment group. The scoring system was developed based on first evaluating all the slides and describing a scoring system specifically to represent this study.

Immunohistochemical co-localization of myosin and luciferase was used to assess the presence and differentiation of donor skeletal muscle-derived mesenchymal stem cells: Briefly, slides were deparafinized and rehydrated to DI water through xylene and a series of Ethanol (100%, 95%, 75%, 50%, DI). Antigen retrieval was done by microwaving the slides just to boiling in Epitope Retrieval Solution (IHC World) and allowed to sit for 10 minutes in the hot solution. The slides were cooled in running DI water and transferred to PBS. The sections were incubated in 1% Hydrogen peroxide for 5 minutes, washed 3 times in TBS-T (Tris Buffered Saline+0.05% Tween 20) and incubated in 5% BSA for 30 minutes before adding anti-luciferase antibody (1:100, Abcam) and incubating overnight at 4° C. Slides were warmed to room temperature, washed 3 times in TBS-T, and secondary biotinylated antibody was added for 30 minutes (1:200 biotin-SP-donkey anti-goat, Jackson ImmunoResearch) at room temperature. The slides were washed 3 times with TBS-T and peroxidase conjugated streptavidin was added (1:500, Jackson ImmunoResearch) for 30 minutes at room temperature. After 3 washes with TBS-T, the peroxidase substrate DAB (diaminobenzindine) was added for 15 minutes, then washed off with DI water, and counterstain was added for 5 minutes. Slides were coverslipped with permount.

There was no difference in body weights of rats between groups at the time of surgery (p>0.05). At euthanasia, however, the mean weight of the DMS/Luc+SMdMSC group (471.4 gm) was significantly less than that of the empty group (521 gm) at the time of euthanasia (p=0.0261) The mean body weight of the DMS group was 506.8 gm. However, all weights are within the normal range for adult Wistar rats. The mean difference in weight between the un-operated TA and the operated TA per group were 0.01157 gm for the empty group, 0.01303 gm for the DMS group and −0.06368 gm for the DMS/Luc+SMdMSC group. There was a statistical difference between the TA difference in the Empty versus the DMS/Luc+SMdMSC group (p=0.0353) and DMS versus the DMS/Luc+SMdMSC group (p=0.0316). There no statistical difference between groups regarding the difference between the weight of the unoperated EDL and the operated EDL.

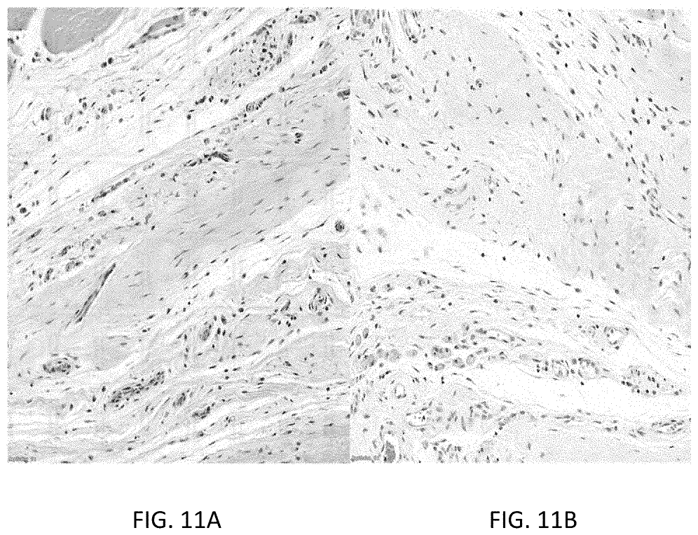

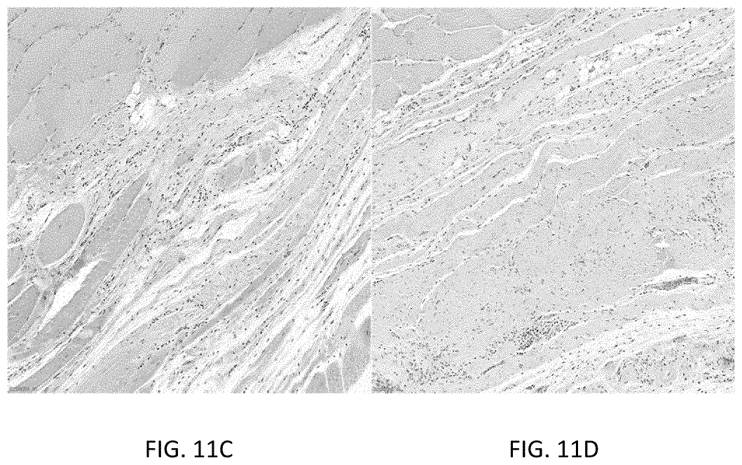

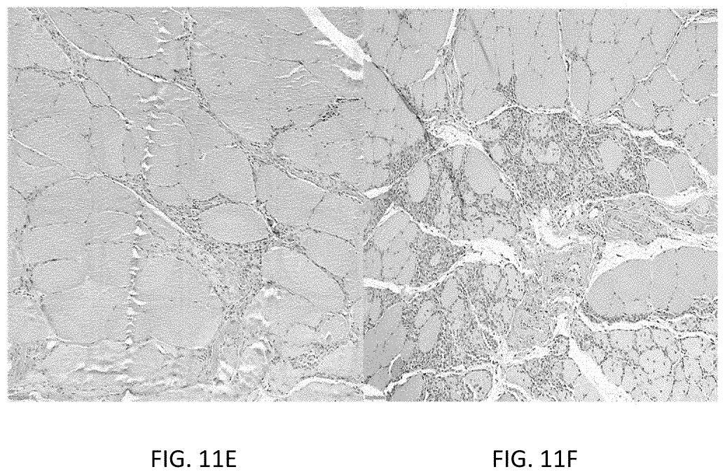

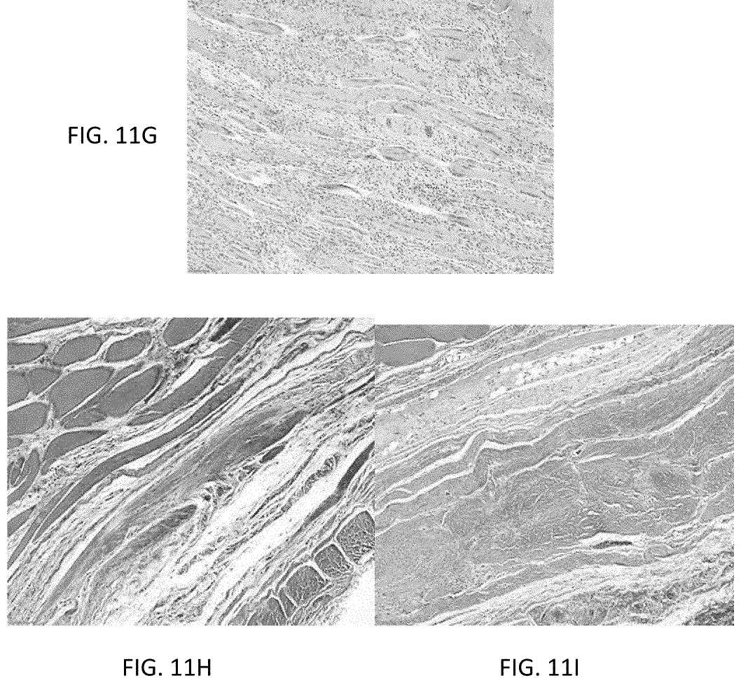

The pathologist developed a scoring system specific for this study. Representative images of scores 1-3 for each group are depicted in FIG. 11A-D.

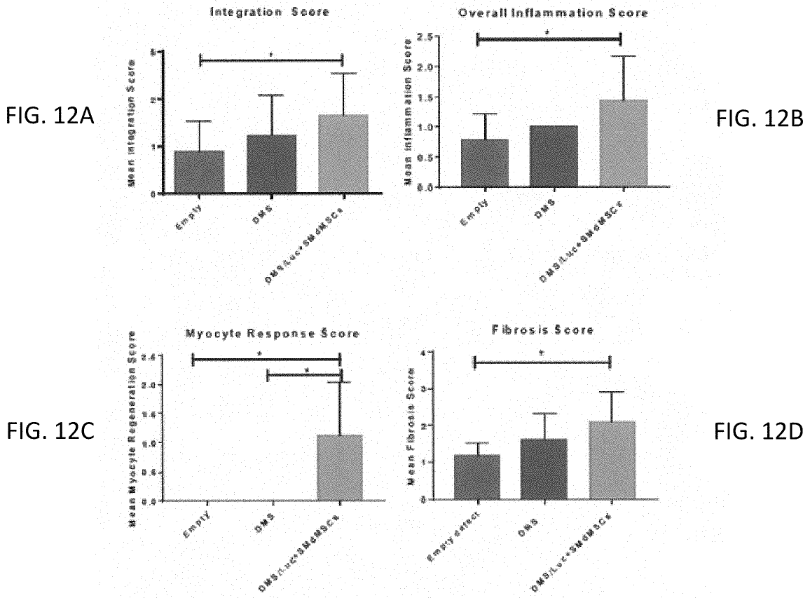

The DMS/Luc+SMdMSC group had a significantly greater mean histologic score of integration, inflammation, myocyte response, and fibrosis as compared to the empty defect group (p<0.05; Table 1, FIG. 12A-D). Cellular infiltrates in the DMS/Luc+SMdMSC group were characterized as lymphocytic (75-100%) with lesser macrophages present (0-25%) and rare mast cells. The DMS/Luc+SMdMSC group also had a greater score of myocyte response than the DMS alone group (p<0.05; Table 1, FIG. 11, FIG. 12C). There were no other significant differences between groups for mean histologic scores of inflammation, integration, fibrosis, or myocyte regeneration (p>0.05). In addition, there was no appreciable scaffold remaining within the defect area.

TABLE 1
Mean Histologic Scores

| | | | Myocyte | |
|---|---|---|---|---|
| | Integration | Inflammation | Response | Fibrosis |
| Empty | .08889 | .7778 | 0 | 1.167 |
| DMS | 1.222 | 1 | 0 | 1.611 |
| DMS/Luc + | 1.667 | 1.444 | 1.111 | 2.111 |
| SMdMSCs | | | | |

Statistical Analysis:

Graph Pad Prism 7 software was used for all statistical analyses. Paired and unpaired t-tests, one-way ANOVA, and Mann Whitney tests were used to compare differences between operated and un-operated contralateral controls. Differences were considered significant when p<0.05.

Conclusions:

There were no animals in the study that experienced illness or complications following implantation of the test articles. Two rats in the study died under anesthesia prior to surgical wound closure. The transplanted Luc+SMdMSCs were identified on BLI immediately following surgery, however, this expression was lost 14 days. Since BLI requires a minimum density this outcome is not surprising as it has been well described in the literature that not all transplanted MSCs survive.

The significantly lower mean body weight in the DMS/Luc+/SMdMSC group as compared to the empty group was an unexpected finding but believed to have no clinical significance as the mean weight was still within the normal range. It could reflect greater activity in the DMS/Luc+/SMdMSC group however, this finding is likely a Type I error. The mean weight difference between the unoperated TA and operated TA at the time of sacrifice was significantly lower in the DMS/Luc+/SMdMSC than the empty group indicating that the DMS/Luc+SMdMSC TA actually weighed more than the unoperated contralateral control TA. This is likely explained by the increased fibrosis and myocyte regeneration present in the DMS/Luc+SMdMSC TA muscle.

Remarkably, the mean peak isometric tetanic force within the TA muscle in the rats that received DMS/Luc+SMdMSCs was the most similar to the contralateral unoperated control TA (FIG. 11). This indicates that, among the groups tested, muscles receiving “re-animated” scaffolds functioned most similarly to normal control muscles. The DMS/Luc+SMdMSC rats also had significantly greater mean scores of integration, inflammation, myocyte regeneration, and fibrosis than the empty VML group (FIG. 11, FIG. 12). Although the score for inflammation was greater in the DMS/Luc+SMdMSC group, the pathologist reported that there was overall minimal to mild inflammation in the tissues. Therefore, despite having the greatest score of inflammation, the DMS/SMdMSC animals had minimal inflammation.

Of greatest interest was that myogenic response noted as indicated by myocyte regeneration and satellite cell proliferation was only present within the DMS/Luc+SMdMSC rats. There was no noted myocyte regeneration or satellite cell proliferation in the other groups, emphasizing that the myogenic response required the presence of the SMdMSCs to occur. In addition the pathologist noted that this regenerative response occurred somewhat distant from the defect rather than within the defect area (FIG. 11E-F, FIG. 12C) which suggests either a migration of transplanted cells out of the defect or native cells toward the defect.

The increased fibrosis within the defect area for the DMS and DMS/Luc+SMdMSc groups as compared to the empty VML group is not surprising. This has been previously reported by Corona et. al. in 2013 wherein when decellularized muscle tissue was implanted using the same VML model, fibrosis occurred, which improved P₀ by ⅓ two months following surgery.⁷ Our scaffold appears to have provided improved functional recovery with our scaffold alone as compared to the Corona paper, however these differences were not significant in the current study.⁷

These findings suggest that the “re-animated” DMS scaffold provided improved functional recovery and integration of the scaffold following VML. While we failed to achieve a statistical difference in P₀, the improved functional outcome for the DMS/Luc+SMdMSCs group and the statistically significant greater histologic scores of scaffold integration, fibrosis, and myocyte response is encouraging and is being used to appropriately power a pivotal study.

Overall, the combination of DMS with Luc+SMdMSCs repair following creation of a volumetric muscle loss injury resulted in, on average, a 97% return to function of the operated TA muscle in comparison to the un-operated control TA as measured by peak isometric tetanic force. This treatment also provided significantly greater integration, fibrosis, and myogenic regeneration than the empty VML group.
1. Busse J W, Jacobs C L, Swiontkowski M F, et al. Complex limb salvage or early amputation for severe lower-limb injury: a meta-analysis of observational studies. J Orthop Trauma 2007; 21:70-76.
2. Patzkowski J C, Owens J G, Blanck R V, et al. Deployment after limb salvage for high-energy lower-extremity trauma. J Trauma Acute Care Surg 2012; 73:S112-115.
3. Egeberg A, Rasmussen M K, Sorensen J A. Comparing the donor-site morbidity using DIEP, SIEA or M S-TRAM flaps for breast reconstructive surgery: a meta-analysis. J Plast Reconstr Aesthet Surg 2012; 65:1474-1480.
4. Sofiadellis F, Liu D S, Webb A, et al. Fasciocutaneous free flaps are more reliable than muscle free flaps in lower limb trauma reconstruction: experience in a single trauma center. J Reconstr Microsurg 2012; 28:333-340.
5. Lu T Y, Lin B, Kim J, et al. Repopulation of decellularized mouse heart with human induced pluripotent stem cell-derived cardiovascular progenitor cells. Nat Commun 2013; 4:2307.
6. Wu X, Corona B T, Chen X, et al. A standardized rat model of volumetric muscle loss injury for the development of tissue engineering therapies. Biores Open Access 2012; 1: 280-290.
7. Corona B T, Ingalls C P. Immediate force loss after eccentric contractions is increased with L-NAME administration, a nitric oxide synthase inhibitor. Muscle Nerve 2013; 47:271-273.

### Example 2

Canine skeletal muscle (appendicular) measuring approximately 3 cm L×1 cm W×1 cm H was decellularized as follows. The muscle was dissected using sterile technique and the weight was recorded. The muscle piece was put into 1% SDS in deionized water in a sterile container and vortexed at room temperature. The SDS solution was changed approximately every 24 hours up to 144 hours. The SDS solution was removed and a wash solution (sterile deionized water) was added. The water was changed out 6 times for 6 washes, approximately every 24 hours. The decellularized muscle scaffold was placed in PBS and refrigerated.

Decellularization of the muscle can be validated with the absence of nuclei on H&E confocal microscopy, and minimal DNA (e.g., less than 50 ng/mg) identified on DNA analysis of the scaffold. SEM can be also performed and indicative that the decellularized scaffold retains its architecture.

Larger samples of muscle tissue can also be decellularized.

### Example 3

Sheep skeletal muscle of various sizes is decellularized as follows. Muscle is dissected using sterile technique and the size and weight is recorded. The muscle piece is put into 1% SDS in deionized water in a sterile container and vortexed at room temperature. The SDS solution is changed approximately every 24 hours up to 144 hours. The SDS solution is removed and a wash solution (sterile deionized water, PBS, etc.) is added. The wash solution is changed out 6 times for 6 washes, approximately every 24 hours. The decellularized muscle scaffold is placed in PBS or other suitable storage buffer and refrigerated.

Decellularization of the muscle can be validated with the absence of nuclei on H&E confocal microscopy, and minimal DNA identified on DNA analysis (e.g., less than 50 ng/mg) of the scaffold. SEM can be also performed and indicative that the decellularized scaffold retains its architecture.

### Example 4

Human skeletal muscle of various sizes is decellularized as follows. Muscle is dissected using sterile technique and the size and weight is recorded. The muscle piece is put into 1% SDS in deionized water in a container and vortexed at room temperature. The SDS solution is changed approximately every 24 hours up to 144 hours. The SDS solution is removed and a wash solution (sterile deionized water, PBS, etc.) is added. The wash solution is changed out 6 times for 6 washes, approximately every 24 hours. The decellularized muscle scaffold is placed in PBS or other suitable storage buffer and refrigerated.

Decellularization of the muscle can be validated with the absence of nuclei on H&E confocal microscopy, and minimal DNA identified on DNA analysis (e.g., less than 50 ng/mg) of the scaffold. SEM can be also performed and indicative that the decellularized scaffold retains its architecture.

All patents and publications mentioned in the specification are indicative of the levels of those skilled in the art to which the present disclosure pertains. All such patents and publications are herein incorporated by reference to the same extent as if each individual publication was specifically and individually indicated to be incorporated by reference.

1. A method for preparing a decellularized muscle scaffold comprising:
(a) providing a muscle sample from a donor and treating the muscle sample with an aqueous solution comprising about 0.5% (w/v) to about 2% (w/v) sodium dodecyl sulfate for greater than 72 hours at room temperature, with agitation, to produce a treated muscle sample, wherein the aqueous solution is replaced about every 20 to 28 hours;
(b) removing the aqueous solution and adding a wash solution to the treated muscle sample, and then agitating for at least about 72 hours at room temperature (“wash period”) to produce a decellularized muscle scaffold, wherein the wash solution is replaced at least six times during the wash period.

2. The method of claim 1, wherein the aqueous solution comprises about 1% (wv) to about 1.5% (wv) sodium dodecyl sulfate.

3. (canceled)

4. The method of claim 1, wherein the aqueous solution does not contain an enzyme.

5. The method of claim 1, wherein the aqueous solution consists of about 1% (wv) to about 1.5% (wv) sodium dodecyl sulfate.

6. (canceled)

7. The method of claim 1, wherein the sample in step (b) is agitated for about 100 hours to about 140 hours.

8. The method of claim 1, wherein the sample in step (b) is agitated for about 120 hours.

9. The method of claim 1, wherein the aqueous solution in step (b) is replaced about every 24 hours.

10. The method of claim 1, wherein the wash period is about 72 hours to about 120 hours.

11. The method of claim 1, wherein the wash solution is replaced at substantially even intervals throughout the wash period.

12. The method of claim 1, wherein the wash solution is sterile water.

13. (canceled)

14. The method of claim 1, wherein the muscle sample was previously frozen.

15. The method of claim 1, wherein the donor is a mammal.

16. (canceled)

17. (canceled)

18. (canceled)

19. (canceled)

20. The method of claim 1, wherein the decellularized muscle scaffold has a DNA content of less than 50 ng of DNA per mg of decellularized muscle scaffold.

21. The method of claim 20, wherein the decellularized muscle scaffold has a DNA content of not more than 40 ng of DNA per mg of decellularized muscle scaffold.

22. (canceled)

23. The method of claim 1, wherein the decellularized muscle scaffold has a preserved microstructure as determined by scanning electron microscopy.

24. The method of claim 1, wherein the method further comprises: (c) adding a sufficient amount of a storage buffer to the decellularized muscle scaffold and storing at about 4° C.

25. A decellularized muscle scaffold produced by a method of claim 1.

26. The decellularized muscle scaffold of claim 25, wherein the decellularized muscle scaffold is seeded with a plurality of muscle stem cells.

27. Use of a decellularized muscle scaffold of claim 25 to treat a subject with damaged or lost muscle.

28. Use of a decellularized muscle scaffold of claim 26 to treat a subject with damaged or lost muscle.

29. Use of a decellularized muscle scaffold of claim 26 to replace damaged or lost muscle in a subject in need thereof.
